(19) United States
(12) Patent Application Publication
(10) Pub. No.: US 2009/0043266 A1
(43) Pub. Date: Feb. 12, 2009

(54) SYRINGE BARREL AND METHOD FOR THE PRODUCTION OF A SYRING BARREL

(75) Inventors: Wolfgang Heidl, Muennerstadt (DE); Hendrik Gebauer, Delitzsch (DE); Lars Richter, Kossa (DE)

Correspondence Address: LUCAS & MERCANTI, LLP, 475 PARK AVENUE SOUTH, 15TH FLOOR, NEW YORK, NY, 10016, US

(73) Assignee: MGLAS AG, Muennerstadt (DE)

(21) Appl. No.: 12/188,520

(22) Filed: Aug. 8, 2008

(30) Foreign Application Priority Data
Aug. 9, 2007 (DE) 10 2007 037 565.6

(51) Int. Cl. A61M 5/34

(52) U.S. Cl. 604/240; 264/482

(57) ABSTRACT

This invention relates to a syringe barrel (1) which, at least in part, has a cylindrical portion (2), whereby a needle (4) is fixed in an axial end portion (3) of the cylindrical portion (2), whereby the needle (4) is arranged in a receiving channel (5) in the axial end portion (3) of the cylindrical portion (2) and whereby the needle (4) is fixed in the receiving channel (5) in at least one fixing area (6, 7) using material of the cylindrical portion (2) which, after melting, bonds around the circumference of the needle (4), seals it and forms a bond between the cylindrical portion (2) and the needle (4). In order to achieve that the needle is fixed more reliably in the syringe barrel and to minimize the dead space volume for drug in the syringe barrel as, well as to improve sterilization, the invention provides that there are at least two fixing areas (6, 7) arranged at an axial distance (a) which fix the needle (4) in the axial end portion (3) of the cylindrical portion (2). Furthermore the invention relates to a method for producing a syringe barrel.

The invention relates to a syringe barrel which, at least in part, has a cylindrical portion, whereby a needle is fixed in an axial end portion of the cylindrical portion, whereby the needle is arranged in a receiving channel in the axial end portion of the cylindrical portion and whereby the needle is fixed in the receiving channel in at least one fixing area using material of the cylindrical portion which, after melting, bonds around the circumference of the needle, seals it and such forms a bond between the cylindrical portion and the needle. This invention further relates to a method for producing a syringe barrel.

Syringe barrels and methods for producing them are well known from the state of the art. DE 101 08 958 A1 describes a method with which a needle is fixed in a cylindrical portion, made of glass, of the syringe barrel, and namely in its axial end portion, by melting glass material in the axial end portion with a laser. The molten glass bonds around the circumference of the needle. During the cooling-off (solidifying) process, the molten glass forms a tight bond between the cylindrical portion of the syringe barrel and the needle.

The advantage of this method is that it is not necessary to use adhesive to fix the needle in the cylindrical barrel. The use of adhesive is particularly questionable in view of the fact that it may affect the drug to be injected.

What is of a disadvantage, however, is that under unfavorable production conditions the bond formed between the cylindrical portion of the syringe barrel and the needle is not always sufficiently strong. In particular this is the case if such bond has to absorb a bending moment that is perpendicular to the longitudinal axis of the needle. In this case the fixing area, which is rather short in the axial direction, has to support the bending moment.

Another problem is that, for construction reasons, there is a certain dead space volume in the axial end portion of the needle, which faces the inside area of the cylindrical portion and which receives the drug to be injected prior to the actual injection. This volume cannot be fully ejected during the injection, which is especially disadvantageous if very costly drugs are administered.

Finally it has been observed, though rarely, that occasionally the needle drops out of the syringe barrel, which points to potential problems that occurred when the needle was melted into the glass material.

Thus, the object of the invention is to further develop a syringe barrel of the mentioned kind and type as well as an associated method for producing it in such a way that the said disadvantages are rectified or at least reduced. The objective therefore is to improve the support of the needle in the axial end portion of the cylindrical portion of the syringe barrel, so that bending moments can be absorbed more reliably. Furthermore the aim is to reduce the dead space volume, so that a higher share of the drug received by the cylindrical portion can be injected. Another aim is to improve the sterilization of the syringe barrel. And finally, the objective is to improve the positioning and support of the needle in the cylindrical portion of the syringe barrel, so that the risk of the needle dropping out is reduced.

The solution of this aim with the invention is characterized in that there are at least two fixing areas arranged at an axial distance, which fix the needle in the axial end portion of the cylindrical portion.

Preferably there are two fixing areas, one of which is arranged at the end of the axial end portion of the cylindrical portion. The fixing area arranged at the end of the axial end portion of the cylindrical portion may have a conical surface or a convex surface at the outer circumference.

Furthermore there may be two fixing areas, one of which is arranged at the axial end of the needle which faces the inside area of the cylindrical portion. In this case it has proven to be of advantage if the inside space of the syringe barrel increases in diameter from the fixing area at the axial end of the needle onward, whereby the surface of the fixing area has an even shape.

Alternatively, the surface of the fixing area may be of a domed, in particular convex, shape.

Preferably the surface of the fixing area is flush with the axial end of the needle, which minimizes the dead space volume. An alternative solution provides that the axial end of the needle slightly protrudes above the surface of the fixing area and faces the inside area of the cylindrical portion.

The syringe barrel is preferably, but not necessarily, made of glass.

The method for producing a syringe barrel of the said kind and type kind is characterized in that the melting and cooling-off processes take place in at least two fixing areas of the axial end portion of the cylindrical portion arranged at an axial distance.

The melting is preferably carried out with a laser, whereby either an Nd:YAG laser, a diode laser or a CO₂ laser can be used.

The melting and cooling-off of the material in the at least two fixing areas can take place sequentially, i.e. in timed sequence.

Alternatively however, it is also possible to let the melting and cooling-off of the material in the at least two fixing areas take place simultaneously.

The laser light can be directed onto the fixing area using focusing optics in such a way, that a circular ring-shaped heating zone forms around the needle.

Alternatively it can be provided that during the melting of the material of the fixing areas, the laser beam is stationary and the syringe barrel rotates such that a ring-shaped heating zone forms. In kinematic reversal, it can—of course—also be provided that during the melting process the syringe barrel is stationary and the laser beam is moved such that a ring-shaped heating zone forms around the needle.

The proposed solution ensures that a very strong bond between the needle and the cylindrical portion of the syringe barrel is formed. The risk of the needle dropping out is significantly reduced, as now at least two fixing areas jointly ensure that the needle is held in place in the syringe barrel.

Furthermore the aim of the invention is to improve the sterilization of the syringe barrel. All sterilization methods known can be used.

Due to the preferred design of the axially positioned internal fixing area, the dead space volume can be reduced substantially, which is of advantage when expensive drugs have to be injected.

Finally the bending moments, too, are absorbed a lot more reliably, as the two (or the at least two fixing areas) axially distanced fixing areas keep the forces acting on the respective supporting points to a minimum.

The drawing illustrates embodiments of the invention as follows:

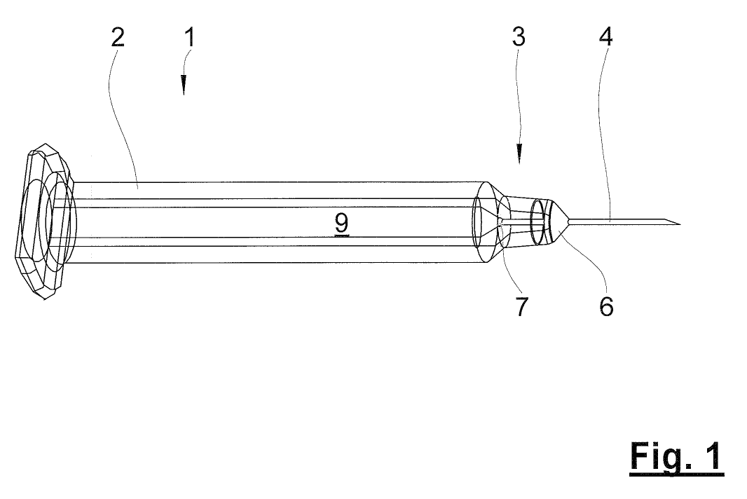

FIG. 1 is a perspective view of a syringe barrel with a cylindrical portion and a needle,

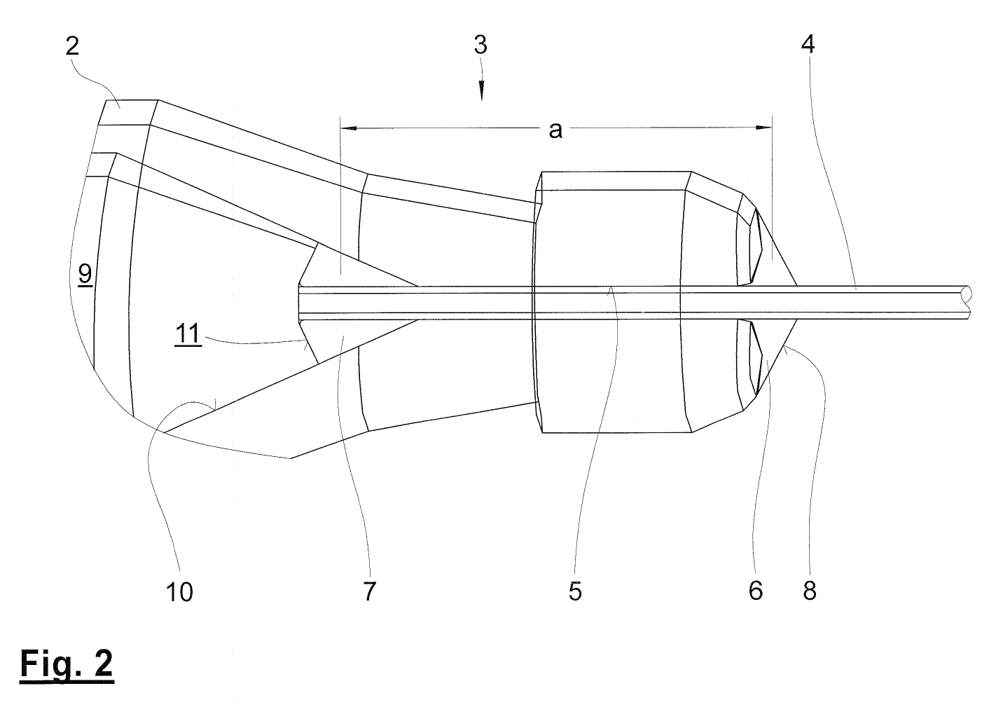

FIG. 2 is a cut through the axial end portion of the cylindrical portion of the syringe barrel with a fixed needle,

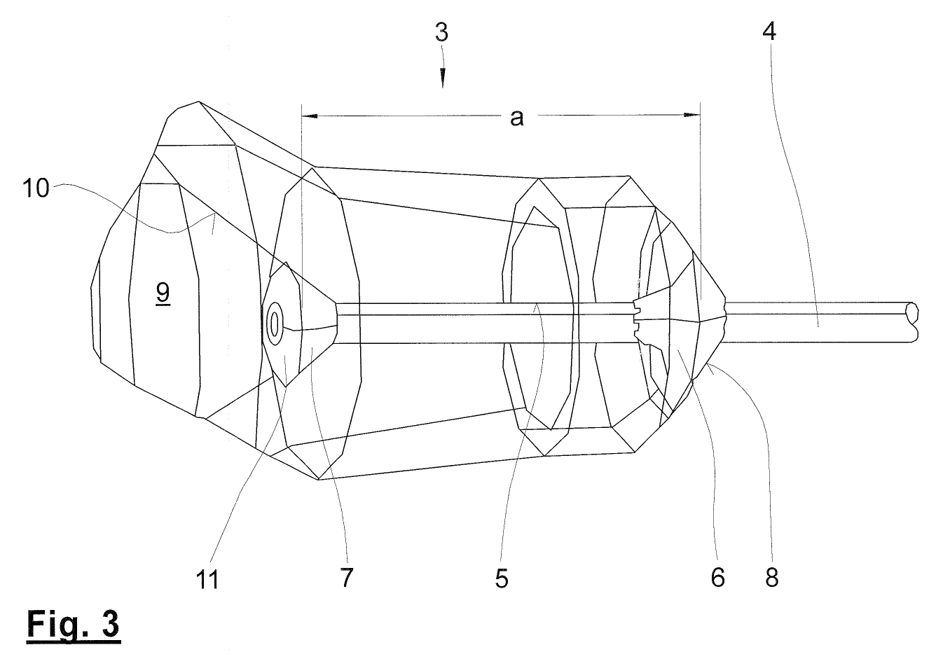

FIG. 3 is a perspective view of the arrangement in accordance with FIG. 2,

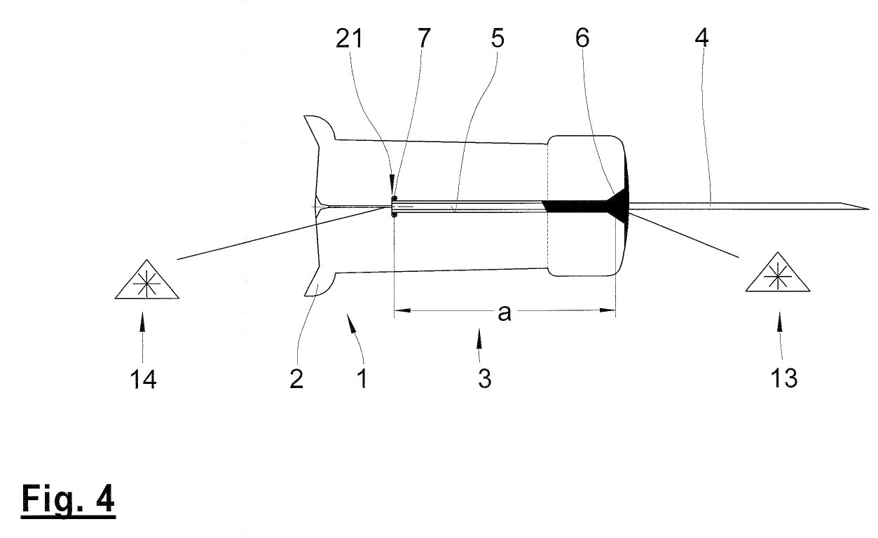

FIG. 4 shows the axial end portion of the cylindrical portion of the syringe barrel with a fixed needle in accordance with an alternative embodiment,

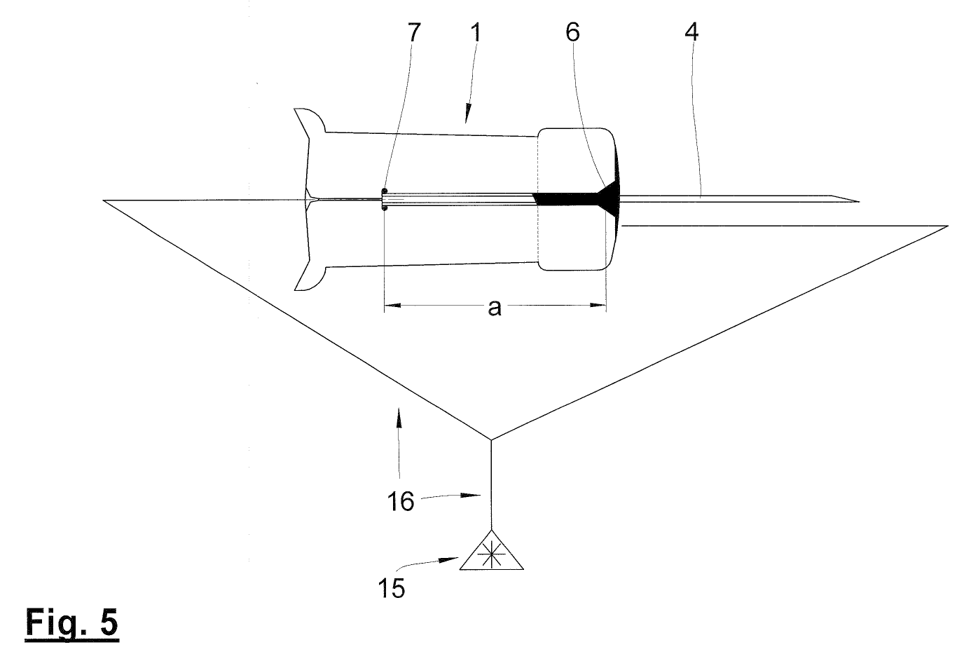

FIG. 5 is a schematic view of how the arrangement is produced in accordance with FIG. 4 using a beam splitter for a laser beam, and

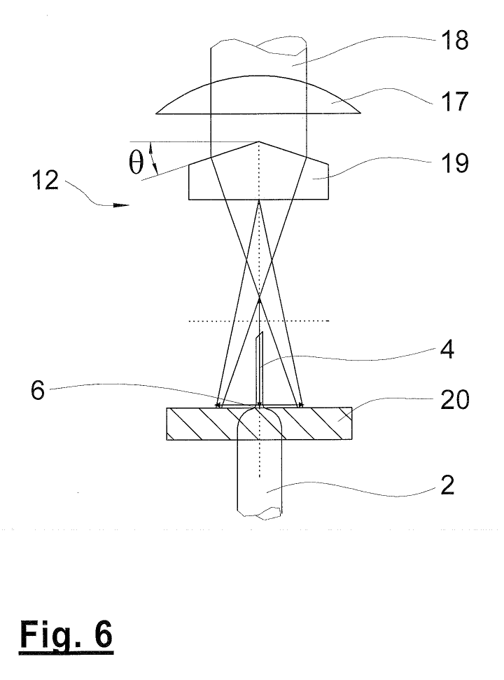

FIG. 6 shows a focusing optics for a laser beam to create a circular ring-shaped heating zone.

FIG. 1 shows a syringe barrel 1 whose main part is a cylindrical (or hollow cylindrical) portion 2, which has an inside space 9 that receives the drug to be injected. In a conventional manner, a plunger, which is not depicted here, and its axial movement inject the drug in the inside space 9. This takes place in a conventional manner too, for instance intravenous with a needle 4, which is fixed in the axial end portion 3 of the cylindrical portion 2 in the syringe barrel 1.

According to the said invention, the needle 4 is fixed by (at least) two fixing areas 6 and 7, arranged at an axial distance a, in the axial end portion 3 of the cylindrical portion 2 (see FIGS. 2 and 3). The axial distance a is preferably 3 to 60 times or at least 3 to 30 times the outer diameter of the needle 4, and in particular preferably 5 to 22 times thereof.

Details of a preferred embodiment of the invention are shown in FIGS. 2 and 3.

Here it is evident that in the axial end portion 3 of the cylindrical portion 2, a receiving channel 5 designed as a cylindrical borehole—has been integrated. The needle 4 is pushed into the receiving channel 5 until the depicted relative position between the end portion 3 and the needle 4 has been reached.

Then a laser is used to melt the fixing areas 6 and 7, so that the molten glass in the areas 6 and 7 bonds around the circumference of needle 4. After the molten glass has cooled off and solidified, there is a tight bond between the needle 4 and the glass barrel in the two fixing areas 6 and 7, which are designed as fixing points.

As regards the embodiments, the melting and solidifying processes take place in accordance with FIGS. 2 and 3 in such a way that a conical surface 8 forms in the fixing area 6.

The second fixing area 7 is located in an area that increases in diameter (tapered or conical shape) and where the axial end portion 3 merges into the cylindrical portion 2. I.e. here, in the inside space 9 of the cylindrical barrel and the axial end portion 3 respectively, a conical shape 10 is present.

After the melting and solidifying processes, the fixing area 7 is formed such that a preferred convex surface 11 is created, which faces the inside space 9. As can be seen farther, the axial (left) end of the needle 4 is flush with the surface 11 of the fixing area 7. Alternatively, however, it is also possible that the needle 4 slightly protrudes from the molten glass material and faces the inside space 9 of the cylindrical portion 2.

Due to this design, the dead space volume in the glass barrel, i.e. the drug volume which cannot be ejected from the syringe, is minimized. The plunger (not depicted) can be designed accordingly congruent to the surface 11.

Furthermore it is immediately evident that, due to the axial distance a provided for between the two fixing areas 6 and 7, bending moments applied by the needle 4 can be absorbed a lot more reliably than this is possible in the state of the technology which provides just one fixing area.

FIG. 4 is a schematic view of how the two fixing areas 6 and 7 are produced. Here two lasers, 13 and 14, have been provided for, each of which heats one of the two fixing areas 6 and 7 and melts the glass material there.

As regards the solution in accordance with FIG. 4, it should be pointed out that here the glass barrel is of an alternative design, because the receiving channel 5 is not of a continuous design but—coming from the outside—extends across a certain axial length and then merges into a borehole with a reduced diameter, through which the drug is injected.

Accordingly the needle 4 is pushed into the axial end portion 3 of the cylindrical portion 2 of the syringe barrel until it reaches the dead stop 21 and fixed there as described above by way of lasers 13 and 14.

The solution in accordance with FIG. 5 provides that not two lasers but just one single laser 15 is used whose light, however, is split by a beam splitter 16, of which there is a schematic view only. The light is directed onto the two spots to be melted to produce the fixing areas 6 and 7.

A solution to melt a glass ring area around the needle 4, which is preferable from the production-technological point of view, is shown in FIG. 6. Here a focusing optics 12 is shown into which a laser beam 18 enters and is directed to a focusing lens 17. By way of a prism (with prism angle Θ) the light is bundled and directed onto the workpiece 20, where a ring-shaped area forms which can be melted by a laser beam 18.

This device called “Axicon” makes it possible to melt glass in the shape of a ring, and it thus represents an alternative to performing a relative turn between the glass barrel and the laser beam.

The aim is always to melt the glass material partially and to the most limited extent possible only, so to prevent loss of shape at the syringe barrel.

Apart from the CO₂ lasers it is also possible to use solid-state lasers. The latter send a laser beam through the glass which heats the needle inside the glass barrel and the glass material around the needle respectively. A CO₂ laser beam, however, cannot fully penetrate the glass.

The power of the laser used is controlled, so that the temperature of the welded spot remains constant after having been heated strongly at a high temperature gradient. Accurate control of the temperature results in a high degree of reproducibility of the process.

As regards details to the laser technology in question, explicit reference is made to DE 198 39 343 A1.

### LIST OF REFERENCES

1 Syringe barrel

2 Cylindrical portion

3 Axial end portion

4 Needle

5 Receiving channel

6 Fixing area

7 Fixing area

8 Conical surface

9 Inside of the cylindrical portion/inside space

10 Conical shape

11 Surface of the fixing area

12 Focusing optics

13 First laser

14 Second laser

15 Laser

16 Beam splitter

17 Focusing lens

18 Laser beam

19 Prism

20 Workpiece

21 Dead stop

a Axial distance

Θ Angle

1. Syringe barrel comprising: a cylindrical portion, whereby a needle is fixed in the axial end portion of the cylindrical portion, whereby the needle is arranged in a receiving channel in the axial end portion of the cylindrical portion and whereby the needle is fixed in the receiving channel in at least one fixing area using material of the cylindrical portion which, after melting, bonds around the circumference of the needle, seals it and such forms a bond between the cylindrical portion and the needle wherein there are at least two fixing areas, arranged at an axial distance, which fix the needle in the axial end portion of the cylindrical portion.

2. Syringe barrel according to claim 1, wherein there are two fixing areas one of which is arranged at the end of the axial end portion of the cylindrical portion.

3. Syringe barrel according to claim 2, wherein the fixing area, arranged at the end of the axial end portion of the cylindrical portion, has a conical surface at the outer circumference.

4. Syringe barrel according to claim 2, wherein the fixing area, arranged at the end of the axial end portion of the cylindrical portion, has a convex surface at the outer circumference.

5. Syringe barrel according to claim 1, wherein there are two fixing areas, one of which is arranged at the axial end of the needle which faces the inside of the cylindrical portion.

6. Syringe barrel according to claim 5, wherein the inside space of the glass barrel increases in diameter from the fixing area at the axial end of the needle onward, whereby the surface of the fixing area has a flat shape.

7. Syringe barrel according to claim 5, wherein the inside space of the glass barrel increases in diameter from the fixing area at the axial end of the needle onward, whereby the surface of the fixing area has a domed shape.

8. Syringe barrel according to claim 7, wherein the domed shape is convex.

9. Syringe barrel according to claim 6, wherein the surface of the fixing area is flush with the axial end of the needle.

10. Syringe barrel according to claim 1, wherein the cylindrical portion is made of glass.

11. Syringe barrel according to claim 1, wherein the axial distance between the two fixing areas is 3 to 60 times, the outer diameter of the needle.

12. Method for producing a syringe barrel comprising: inserting a needle into a receiving channel in the axial end portion of an at least partially cylindrical portion of the syringe barrel melting material of the axial end portion of the cylindrical portion is melted in at least one fixing area by a heat source, cooling the material so that it bonds around the needle, seals it and forms a bond between the axial end portion of the cylindrical portion and the needle, wherein the melting and cooling processes take place in at least two fixing areas of the axial end portions of the cylindrical portion arranged at an axial distance.

13. Method according to claim 12, wherein the heat source emits electro-magnetic radiation.

14. Method according to claim 12, wherein the melting is carried out with a laser.

15. Method according to claim 14, wherein the melting is carried out with an Nd:YAG laser or a diode laser.

16. Method according to claim 14, wherein the melting is carried out with a CO₂ laser.

17. Method according to claim 12, wherein the melting and cooling of the material in the at least two fixing areas takes place sequentially.

18. Method according to claim 12, wherein the melting and cooling of the material in the at least two fixing areas takes place simultaneously.

19. Method according to claim 14, wherein the laser light is directed onto the fixing areas by way of focusing optics such that a circular ring-shaped heating zone forms there.

20. Method according to claim 14, wherein during the melting of the material of the fixing areas, the laser beam is fixed and the glass barrel rotates such that a ring-shaped heating zone forms.

21. Method according to claim 14, wherein during the melting of the material of the fixing areas, the glass barrel is fixed and the laser beam is moved such that a ring-shaped heating zone forms
